(19) United States
(12) Patent Application Publication
(10) Pub. No.: US 2008/0114334 A1
(43) Pub. Date: May 15, 2008

(54) Adhesive Marker

(76) Inventor: James W. Voegele, Cincinnati, OH (US)

Correspondence Address: FROST BROWN TODD LLC, 2200 PNC Center, 201 East Fifth Street, Cincinnati, OH, 45202, US

(21) Appl. No.: 11/558,492

(22) Filed: Nov. 10, 2006

(51) Int. Cl. A61M 5/00; A61B 17/32

(52) U.S. Cl. 604/518; 606/167

(57) ABSTRACT

A method for creating an identifying mark on tissue within a patient is disclosed. The method comprises the step of first positioning a surgical marker device within the patient's body. Next, the tissue is studied and identified, and one or more visible marks are drawn on the internal tissue. The visible the marks comprise a mix of a visible pigment and a polymer adhesive that is selected from the group consisting of a polymerizable monomer, a polymerizable 1,1,1,1-disubstituted ethylene monomer, and a cyanoacrylate. The visible marks are adhered to the tissue by polymerizing the polymer adhesive with tissue contact. Additionally, a method of excising diseased tissue along a series of marks drawn on internal tissue is disclosed

### FIELD OF THE INVENTION

The present invention relates, in general, to surgical marking devices and methods, and in particular to methods of using an adhesive marking device.

### BACKGROUND OF THE INVENTION

During endoscopic or open surgery, a surgeon must first gain access to the surgical site and then must spend a good amount of time studying, reviewing, turning and moving the tissue and organs about. The reviewing process is to familiarize the surgeon with each patients distinctive anatomy, enable the surgeon to identify critical areas to avoid such as major arteries, nerve clusters, the urethra, and the like, and to assess the disease state requiring treatment. Then, the surgeon plans exactly where the surgery will occur, and select which tools to use. All of this information is retained in the surgeon's memory.

Consequently, a significant need exists for a method of using an adhesive marking device that can place biocompatible roadmaps or marks within a patient, reduce the need for an expansive memory, mark critical areas of tissue to avoid, mark excision lines, can be easily applied onto and attached to moist body organs, and can be viewed from outside of the body.

### BRIEF SUMMARY OF THE INVENTION

The invention overcomes the above-noted and other deficiencies of the prior art by providing a method for creating an identifying mark on tissue within a patient. The method comprises a first step of positioning a surgical marker device within the patient's body. A second step comprises identifying and studying internal tissue within the patient's body. A third step comprises marking internal tissue with the surgical marker device to indicate areas of interest by drawing one or more visible marks on the internal tissue. The one or more visible marks comprise a mix of a visible pigment and a polymer adhesive that is selected from the group consisting of a polymerizable monomer, a polymerizable 1,1,1,1-disubstituted ethylene monomer, and a cyanoacrylate. A fourth step comprises adhering the mark to tissue by polymerizing the polymer adhesive with tissue contact.

In one aspect of the invention, a method for excising diseased tissue from a patient's body is disclosed. The method comprises the first step of positioning a surgical marker device in the patient's body. The second step comprises identifying diseased tissue areas within the patient's body. The third step comprises applying one or more marks about the diseased tissue within the patient. The one or more marks are comprised of a pigment and a polymer adhesive selected from the group consisting of a polymerizable monomer, a polymerizable 1,1,1,1-disubstituted ethylene monomer, and a cyanoacrylate about the diseased tissue. The next step is adhering the one or more marks to tissue by polymerizing the polymer adhesive with tissue contact. And, the last step is excising the diseased tissue along the one or more marks.

These and other objects and advantages of the present invention shall be made apparent from the accompanying drawings and the description thereof.

### BRIEF DESCRIPTION OF THE FIGURES

The accompanying drawings, which are incorporated in and constitute a part of this specification, illustrate embodiments of the invention, and, together with the general description of the invention given above, and the detailed description of the embodiments given below, serve to explain the principles of the present invention.

FIG. 1 is an isometric view of a surgical adhesive marker device marking cut lines about a lung tumor.

FIG. 2 is a side view of a stomach marked with an adhesive marker to indicate cut and staple lines for a bariatric procedure.

FIG. 3 is a side view of the stomach of FIG. 2 after the stomach has been resected.

FIG. 4 is a view of a lower portion of anatomy referred to as the triangle of doom showing a hernia and a surgeon using an adhesive marker to mark a circle about a safe attachment point for the attachment of a hernia mesh.

FIG. 5 is a view of the triangle of doom of FIG. 4 showing four safe attachment points identified and marked with circles from the adhesive marker and showing pads of collagen being placed within the safe attachment points.

FIG. 6 shows the view of FIG. 5 with a hernia mesh being placed over the hernia prior to attaching the mesh to the collagen pads within the safe attachment points.

FIG. 7 is a view of a stomach with a tumor with cut lines marked with the adhesive marker.

FIG. 8 is the view of FIG. 7 with the tumor excised and the cut lines painted with an adhesive marker containing drugs, and a pair of arrows marked onto the stomach with a second adhesive marker containing an image contrasting agent such as barium.

### DETAILED DESCRIPTION OF THE INVENTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

During endoscopic or open surgery, a surgeon must first gain access to the surgical site and then must spend a good amount of time studying, reviewing, turning and moving the tissue and organs about. The reviewing process is to familiarize the surgeon with each patients distinctive anatomy, enable the surgeon to identify critical areas to avoid such as major arteries, nerve clusters, the urethra, and the like, and to assess the disease state requiring treatment. Then, the surgeon plans exactly where the surgery will occur, and select which tools to use. All of this information is retained in the surgeon's memory.

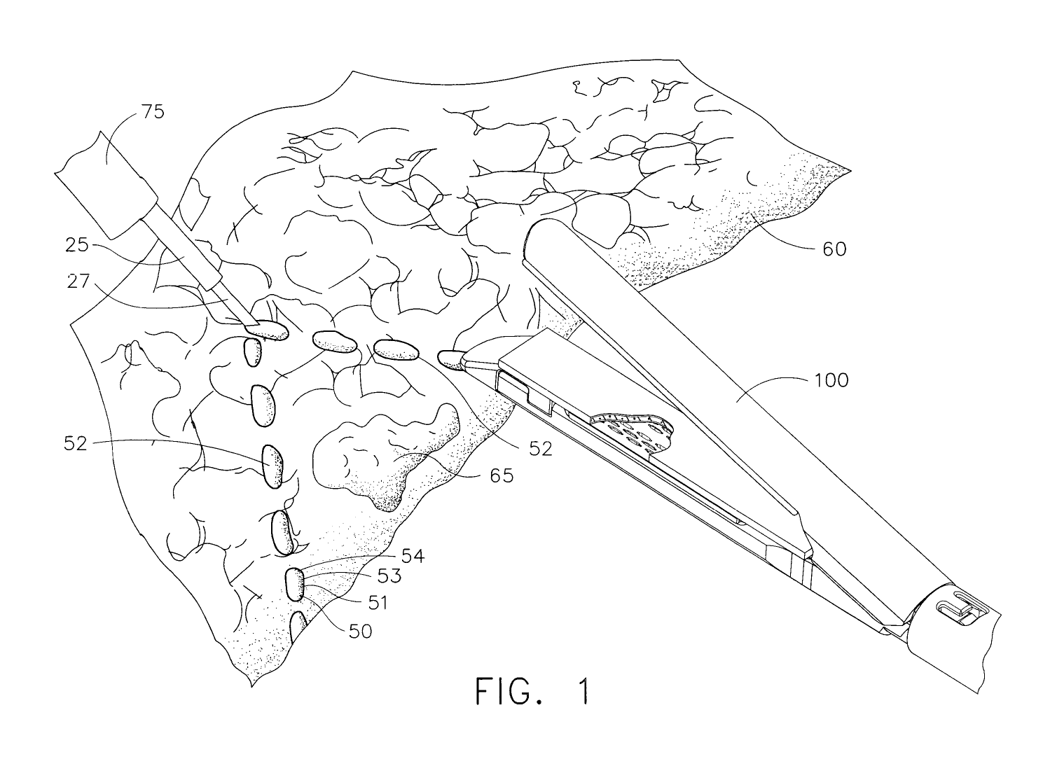

FIG. 1 illustrates an adhesive marker device 25 that is applying an adhesive mark 50 onto tissue during the reviewing process by the surgeon. As shown, the surgeon has accessed the thoracic cavity, has collapsed the lung 60 to create operative space, and has identified a cancer or melanoma 65 in the patient's lungs. The surgeon is using the adhesive marker device 25 to quickly mark the location of melanoma 65 by placing adhesive marks 50 about the identified melanoma 65 in the patient's lungs. The surgeon has used a dashed line 52 to mark cut lines that identify safe margins around the melanoma 65 and are placed to avoid major vascular structures. Once the visible lines 52 are applied, the surgeon can easily move on and to inspect the remainder of the lung 60 confidant that he can quickly and easily go back to this and any other surgical site with the adhesive marks 50. An endocutter 100 is shown within the thoracic cavity and may be used to cut and staple along the visible lines 52 in the process of excising the melanoma 65.

The adhesive mark 50 can be formulated to easily create lines on moist internal organs, mark through bodily fluids and blood at wound sites, and be biocompatible. The adhesive marker 50 of FIG. 1 can be a combination of a base adhesive compound such as a biocompatible polymeric adhesive 51 in combination with one or more other compounds such as biocompatible marker compounds 53 which can include dyes, pigments or colorants in any combination with any other compounds. The polymeric adhesive 51 of the marker can easily be applied to tissue in a fluid unpolymerized state, and can be polymerized by tissue contact, moisture, or adhesive initiators that can polymerize or set the polymer adhesive into a durable mark on the tissue. For the example adhesive marker 50 shown in FIG. 1, the biocompatible marker compounds 53 can be a pigment 54 that is easily visible in natural and endoscopic light for enhanced visualization and recognition.

Thus, an adhesive marker device 25 that can place adhesive marks 50 can offer the surgeon an ability to quickly and easily place visible lines 52 or other marks within a patient to identify cut lines and cut paths, mark melanomas and other areas for pathology, identify and mark body landmarks and anatomical avoidance areas such as large vascular vessels or nerve clusters. Alternately the adhesive marks 50 can be used to identify the shape to be cut prior to the actual cutting of an organ which can distort the tissue such as a stomach during a Roux-en-Y. Additionally, the adhesive marker device 25 that can place adhesive marks 50 can minimize the surgical time needed to re-locate portions of anatomy that have been previously identified and marked, can provide error prevention by marking tissue that should and should not be excised, and can be used as a teaching tool to mark anatomy and identify tissue planes. Thus, using a polymerizable adhesive as a base compound to create a visible adhesive mark 50 could offer new advantages over other conventional marking techniques such as clips, fasteners, barbs, injectables, dyes, graspers clamped on tissue and the like.

In yet another alternate embodiment of the disclosure, the adhesive mark 50 can be foamed with a foaming agent to produce bubbles 58 therein or from a chemical reaction.

Alternately, for example, the adhesive mark 50 can be formulated to be absorbable within the body. Suitable absorbable adhesives can be found in U.S. Pat. No. 6,620,846 by Jon et al., and U.S. Pat. No. 3,995,641 to Kronenthal et al. both of which are hereby incorporated by reference in their entirety.

### Adhesive Marker Device

In FIG. 1, the adhesive marker device 25 is shown extending out of an endoscope 75. The adhesive marker device 25 can comprise a storage chamber 29 (not shown) filled with the adhesive mark 50, and an application tip 27 that operably couples the storage chamber 29 to the application tip 27 for the dispersing of adhesive marker 50 onto tissue.

The surgical marker device 25 be any one of a number of devices that can be used to dispense, paint, brush, apply, coat, dispense, spray or flow an adhesive marker 50 comprising the base polymeric adhesive 51 in combination with a number of other biocompatible compounds 53. For example, the surgical marker device 25 can be a marking pen, a roller ball marker, a felt tipped marker, a brush marker, a porous tipped marker, a spray, or any other marker device that can apply an adhesive 51 containing one or more additional compounds 53 such as a pigment 54.

The use of polymeric adhesives and adhesive application devices are known in the art such as those described in U.S. Patent Application number 2004019075 by Goodman et al entitled “Applicators, Dispensers, and Methods for Dispensing and Apply Applicators, Dispensers and Methods for Dispensing and Applying Adhesive Materials” which is hereby incorporated by reference in its entirety. Such adhesive application devices are known for their uses to glue tissue together.

### The Base Adhesive of the Adhesive Marker

By way of example, polymeric adhesive 51 could be a single part or a dual part adhesive that is a polymerizable and/or cross-linkable material such as but not limited to a cyanoacrylate adhesive. The adhesive materials, for example, may be a monomeric (including prepolymeric) adhesive composition, a polymeric adhesive composition, or any other compound that can adhere to tissue. In embodiments, the monomer may be a 1,1-disubstituted ethylene monomer, e.g., an alpha.-cyanoacrylate. When cross linked or polymerized, the cyanoacrylate can change from a liquid to a solid. Polymerized adhesives for example, can be formulated to be flexible to rigid. If desired, adhesives 51 can be a single part or dual part adhesive. Polymerization of the adhesive 51 can occur from, but is not limited to, exposure to natural tissue moisture, saline applications or application of an adhesion initiator. Alternately any other polymerizable adhesive 51 can be used as the base adhesive such as but not limited to a polymerizable acrylic, epoxy or silicone.

### Biocompatible Marker Compounds

As stated above, the adhesive marker 50 is a combination of the adhesive 51 in combination with visual compounds 53 such as but not limited to colorants, pigments, dyes, and fluorescent materials. The visual compounds 53 can be visible under visual light, ultraviolet light, and can include compounds that can become visible outside of the patients body with other detection devices such as but not limited to X-rays, MRI, an ultraviolet or an exciter light.

Additionally, for example, one or more alternate compounds 54 can be combined with the adhesive 51 and the visual compounds 53 of the adhesive marker 50. These compounds of the example of the can include, but are not limited to drugs, medicaments, and contrasting agents such as but not limited to barium, or any other commonly used surgical compounds, or any combination thereof. Examples of alternate compounds 54 and combinations thereof can also include, but are not limited to: adhesive initiators, image enhancing agents, necrosing agents, sclerosing agents, coagulants, therapeutic agents, medicaments, analeptic agents, anesthesia agents, antidiuretic agents, analgesic agents, antiseptic agents, antispasmodic agents, cardiac agents, depressant agents, diuretic agents, hemostatic agents, hormonal agents, sedative agents, stimulant agents, vascular agents, time release agents and the like.

### Bariatric Surgery

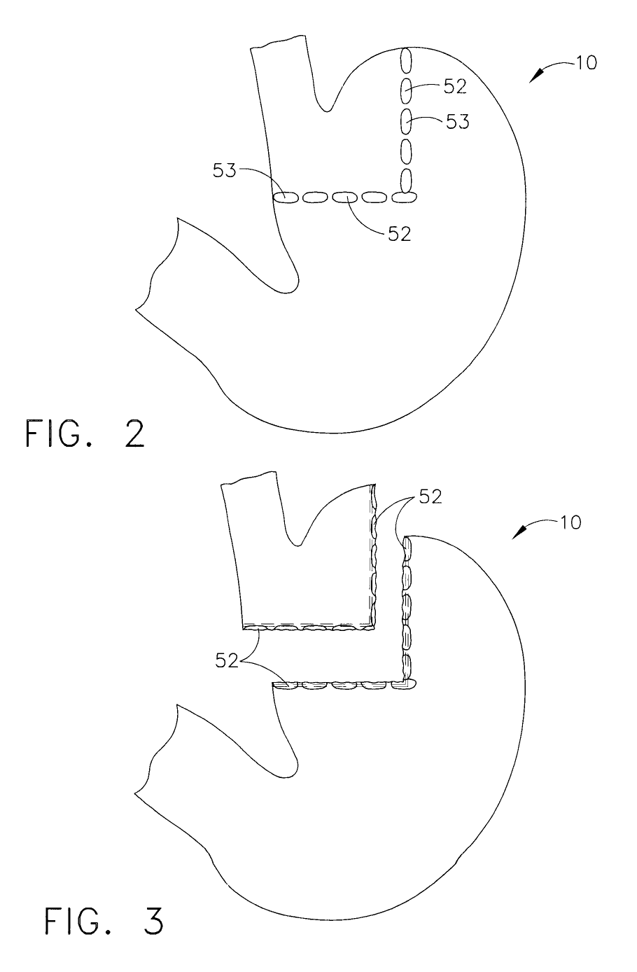

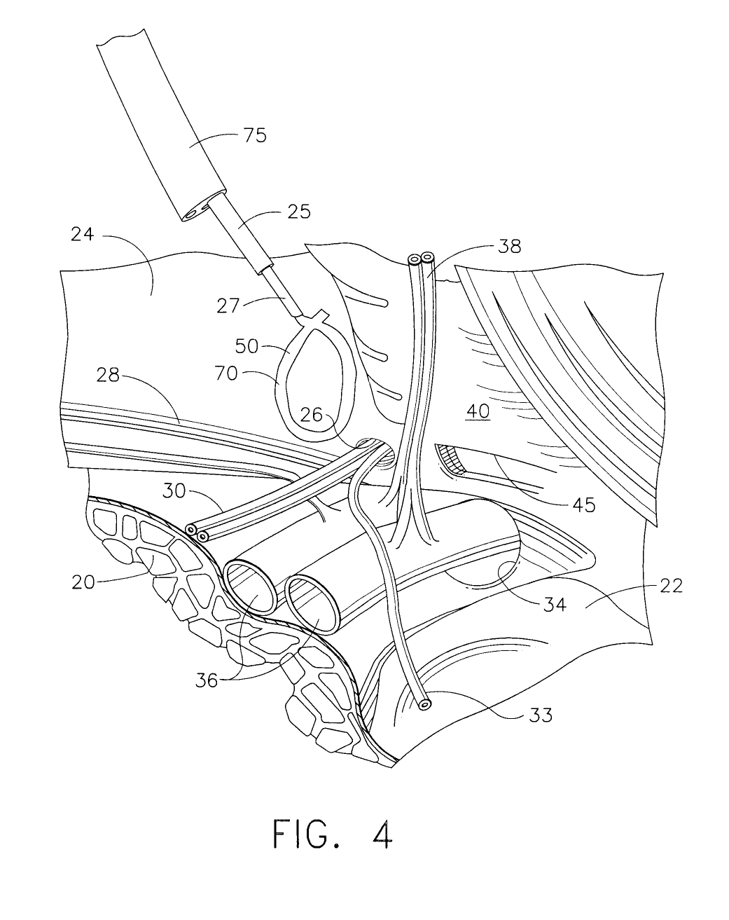

FIGS. 3 and 4 show the first step in a ROUX-en-Y surgical procedure wherein the portion of stomach that is to be resected is marked with the adhesive marker 50. In FIG. 2 the surgeon has placed a pair of dashed lines 52 of an adhesive with a colorant as the visual compound 53. In FIG. 4, the surgeon has resected the tissue with an endocutter 100 along the dashed lines as the first step in the Roux-en-Y surgery.

### Adhesive Marker Use in Hernia Mesh Placement in the Triangle of Doom

FIGS. 4-6 can illustrate the first steps in the repair of a defect 45, such as an inguinal hernia located in inguinal tissue 40 such as the inguinal floor. The delicate anatomical structures of the left inguinal anatomy of a human patient are illustrated in order to particularly point out the usefulness of the present invention.

Generally, the inguinal hernia 45 is accessible through abdominal muscle 20. As can be well appreciated, an extremely sensitive network of vessels and nerves exist in the area of a typical inguinal hernia 45, which requires a surgeon to conduct a hernia repair with great skill and caution. For instance, in the transverse abdominis aponeurosis 24, an internal ring 26 permits gastric vessels 30 and Vas deferens 33 to extend therethrough over an edge of inguinal ligament 28. The Femoral canal 34 is located near Cooper's ligament 22 and contains external iliac vessels 36 and inferior epigastric vessels 38.

In many cases, the edge of the inguinal ligament 28 and Cooper's ligament 22 serve as anatomical landmarks and support structures for supporting surgical fasteners such as those mentioned previously. The area containing the external iliac vessels 36 and the Vas deferens 33 is commonly known to surgeons as the “Triangle of Doom”. Accordingly, it is critical that the surgeon avoid injuring any of these vessels above and extreme care must be taken when performing dissection, suturing or stapling within this area. In FIG. 4, the surgeon has identified a “safe” attachment point for a prosthetic mesh that will be used to repair the defect 45. The surgical marker device 25 is shown extending out of the endoscope 75 and is marking a circle 70 about the first “safe” attachment point in the triangle of doom.

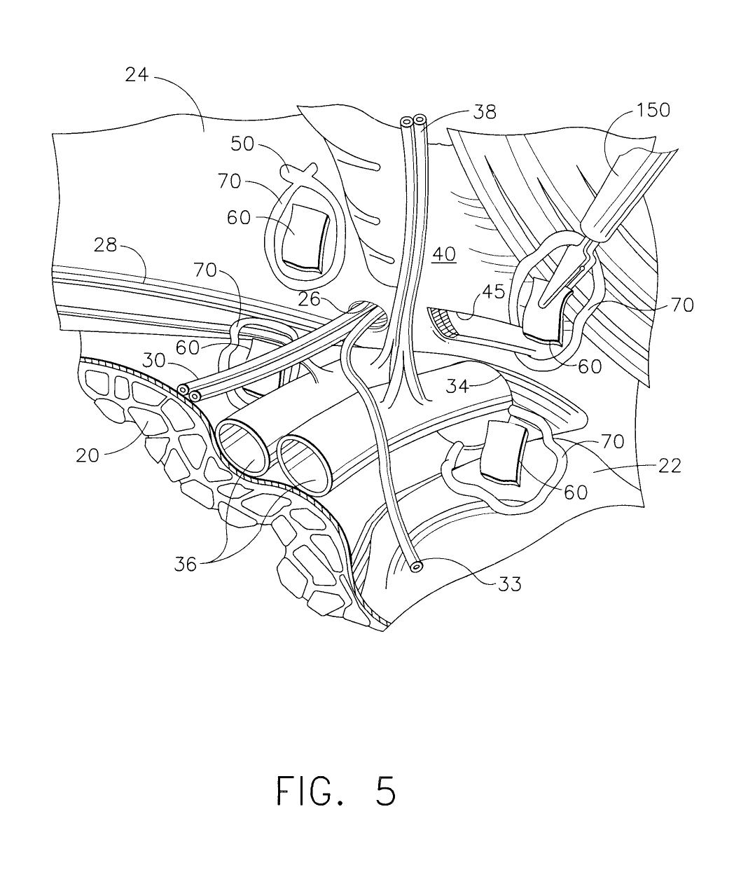

FIG. 5 shows four “safe” attachment points identified and marked with the adhesive marker 50 as a series of circles 70. A grasper 150 is placing a plurality of collagen pads 60 onto “safe” areas marked by circles 70 around the defect 45 on surrounding tissue such as Cooper's ligament 22, the edge of the inguinal ligament 28, the inguinal floor 40, and the transverse abdominis aponeurosis 24.

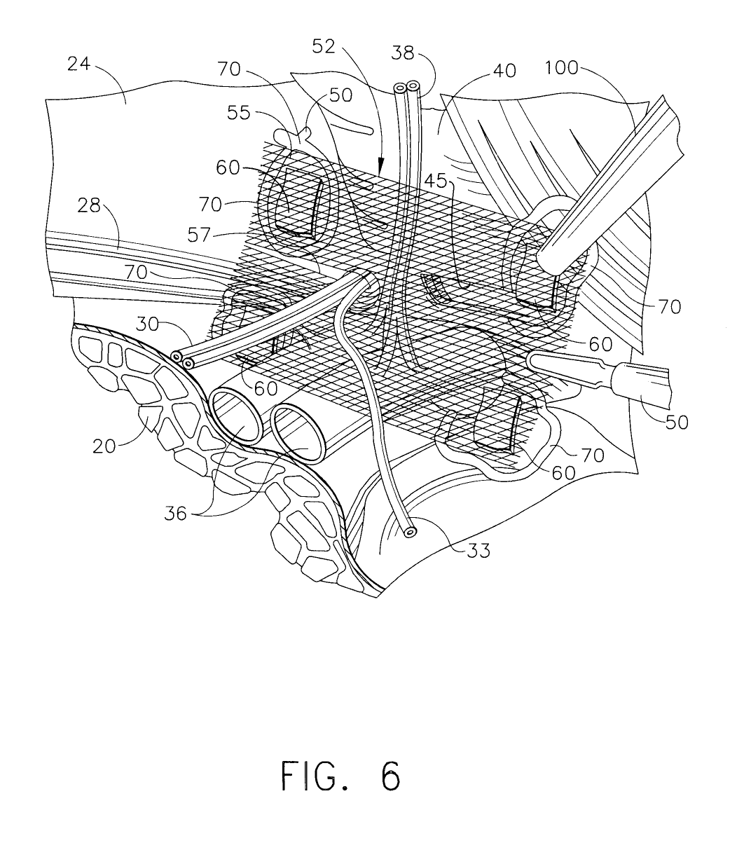

As illustrated in FIG. 6, a patch 55 is being placed over the collagen pads 60 and the circles 70 of adhesive marker 50 prior to adhering the patch to the collagen pads 60 with energy in the treatment of the inguinal hernia. The patch 55 may consist of any desired configuration, structure or material and could be made of PROLENE® (a known polymer made up of fibers) and preferably configured as mesh. It is within the training and comfort zone for surgeons to use the PROLENE® mesh patch 55 since the patch 55 is easily sized, such as providing a side slot 57, for accommodating the gastric vessels 30 and the Vas deferens 33. As illustrated, the patch 55 is placeable over the defect 45 and the collagen pads 60 for providing a sufficient barrier to internal viscera (not shown) of the abdomen which would otherwise have a tendency to protrude through the defect 45 and cause the patient a great deal of pain and discomfort.

The surgical procedure and technique of attaching a mesh pad to tissue in the treatment of an inguinal hernia is taught in great detail in U.S. Pat. No. 5,972,007 by Sheffield et al. entitled “Energy-Base Method Applied to Prosthetics for Repairing Tissue Defects” which is hereby incorporated by reference in its entirety.

### Adhesive Marker and Stomach Carcinoma

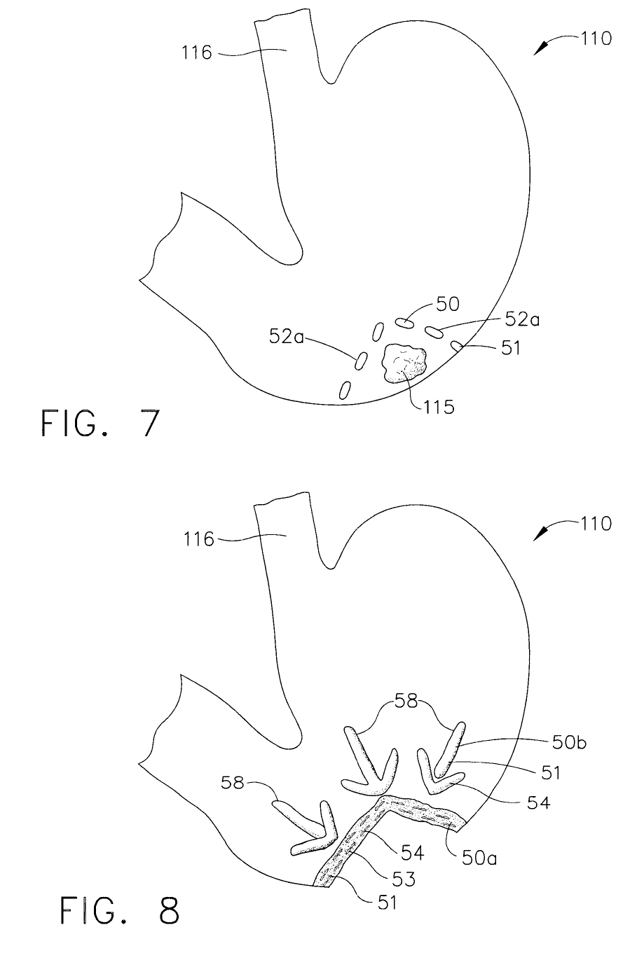

FIGS. 7 and 8 show a patient's stomach 110 and esophagus 116. A carcinoma 115 has been located and a series of adhesive marks 50 in the form of dashed visible lines 52a to indicate the cut line. In FIG. 8, the carcinoma 115 has been excised with an endocutter along the visible lines 52a. The stapled tissue at the excision has been painted with a second adhesive marker 50a comprising the adhesive 51, visual compounds 53 such as a pigment, and alternate compounds 54 such as one or more cancer treating drugs. A third set of lines 52 A set of arrows 58 are marked on the stomach pointing towards the excision site. An adhesive mark 50b comprising an adhesive 51 and a pigment 54 such as barium is used to draw the arrows. The adhesive mark 51b is formulated to resist breaking down within the patient. If the surgeon desires to perform a checkup on the surgical site at a later period, the barium in the adhesive marker 50 can easily be seen outside of the patient in an X-ray and can direct the surgeon's attention to the previous surgical site at a glance.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

While the present invention has been illustrated by description of several embodiments and while the illustrative embodiments have been described in considerable detail, it is not the intention of the applicant to restrict or in any way limit the scope of the appended claims to such detail. Additional advantages and modifications may readily appear to those skilled in the art.

What is claimed is:

1. A method for creating an identifying mark on tissue within a patient, the method comprising:
a) positioning a surgical marker device within the patient's body;
b) identifying and studying internal tissue within the patient's body;
c) marking internal tissue with the surgical marker device to indicate areas of interest by drawing one or more visible marks on the internal tissue, the marks comprising a mix of a visible pigment and a polymer adhesive that is selected from the group consisting of a polymerizable monomer, a polymerizable 1,1,1,1-disubstituted ethylene monomer, and a cyanoacrylate and;
d) adhering the one or more visible marks onto internal tissue by polymerizing the polymer adhesive with tissue contact.

2. The method of claim 1 wherein the step of marking the internal tissue with the surgical marker device includes the step of foaming the polymer adhesive and the pigment with a foaming agent to create a foam mark.

3. The method of claim 1 wherein the visible pigment can be visualized from outside the body and includes the step of visualizing the mark on internal tissue from outside the body.

4. The method of claim 3 including the step of visualizing the mark on internal tissue from outside the body with x-rays.

5. The method of claim 3 including the step visualizing the mark on internal tissue from outside the body with ultrasound.

6. The method of claim 3 including the step of visualizing the mark on internal tissue from outside the body with MRI.

7. The method of claim 1 including the step of marking vascular structures.

8. The method of claim 1 including the step of marking nerve clusters or structures.

9. The method of claim 1 including the step of marking the urethra.

10. The method of claim 1 including the step of marking the vas deferens.

11. The method of claim 1 including the step of marking safe portions of anatomy for surgery.

12. The method of claim 1 including the step of placing a surgical fastener into a marked safe portion of anatomy.

13. A method for excising diseased tissue from a patient's body, the method comprising:
a) positioning a surgical marker device in the patient's body;
b) identifying diseased tissue areas within the patient's body;
c) applying one or more marks about the diseased tissue within the patient, the one or more marks comprising a pigment and a polymer adhesive selected from the group consisting of a polymerizable monomer, a polymerizable 1,1,1,1-disubstituted ethylene monomer, and a cyanoacrylate about the diseased tissue;
d) adhering the one or more marks to tissue by polymerizing the polymer adhesive with tissue contact; and
e) excising the diseased tissue along the one or more marks.

14. The method of claim 13 including the step of marking the site of the excision with a second adhesive marker device, wherein the one or more second marks comprise a mix of the polymerizable adhesive and a therapeutic drug for treatment of tissue at the surgical site.

15. The method of claim 14 including the step adhering the one or more second marks to tissue by polymerizing the polymer adhesive with tissue contact.

16. The method of claim 15 wherein the polymerized adhesive of the one or more second marks is bioabsorbable and includes the step of releasing the drug by gradually absorbing the adhesive of the one or more second marks and the drug.

17. The method of claim 13 including the step of marking the site of the excision within the patients body with a third adhesive marker device, wherein the one or more third marks comprise a mix of the polymer adhesive and a pigment visible through the patients body.

18. The method of claim 17 including the step adhering the one or more third marks third marks to tissue by polymerizing the polymer adhesive with tissue contact.
